SEQUENCE LISTING (1 sequence)

SEQ ID NO: 1 - protein, 158 aa, Artificial Sequence
  Mutein of Human Tear Lipocalin
HHLLASDEEIQDVSGTWYLKAMTVDREFPEMNLESVTPMTLTTLEGGNLEAKVTMLISGRCQEVKAVLEKTDEPGKYTADGGKHVAYIIRSHVKDHYIFYCEGELHGKPVRGVKLVGRDPKNNLEALEDFEKAAGARGLSTESILIPRQSETCSPGSD

(19) United States
(12) Patent Application Publication
(10) Pub. No.: US 2013/0225505 A1
(43) Pub. Date: Aug. 29, 2013

(54) MUTEINS OF HUMAN TEAR LIPCALIN FOR TREATING NEOVASCULAR DISEASE OF THE ANTERIOR SEGMENT OF THE HUMAN EYE

(71) Applicant: ALLERGAN, INC., (US)

(72) Inventor: Michael R. Robinson, Irvine, CA (US)

(73) Assignee: ALLERGAN, INC., Irvine, CA (US)

(21) Appl. No.: 13/680,956

(22) Filed: Nov. 19, 2012

Related U.S. Application Data
(60) Provisional application No. 61/563,304, filed on Nov. 23, 2011.

(51) Int. Cl. C07K 14/435

(52) U.S. Cl. CPC C07K 14/435
USPC 514/20.8

(57) ABSTRACT

Disclosed herein is a method of treating neovascular diseases of the anterior segment of the human eye, the method comprising administering to the eye muteins of human tear lipocalin that target VEGF.

### CROSS-REFERENCE TO RELATED APPLICATIONS

This patent application claims priority to U.S. Provisional Patent Application No. 61/563,304, filed Nov. 23, 2011, the entire contents of both of which are hereby incorporated by reference.

### BACKGROUND

Disclosed herein is a method of treating neovascular diseases of the anterior segment of the human eye, the method comprising administering to the eye muteins of human tear lipocalin that target VEGF. The inventors have unexpectedly discovered that such muteins penetrate the corneal epithelial cell layers of the eye, making them suitable for the treatment of neovascular diseases of the anterior segment of the eye.

### BRIEF DESCRIPTION OF THE DRAWINGS

FIG. 1 shows lipocalin muteins comprising at least two mutated amino acid residues at any sequence position in the N-terminal peptide stretch and the three peptide loops BC, DE, and FG.

FIG. 2 shows amino acids in the three loops at the closed end of the internal ligand binding site of a tear lipocalin and/or the N-terminal peptide stretch of the tear lipocalin that can be mutated in order to obtain lipocalin muteins that bind VEGF with determinable affinity.

FIG. 3 shows the front of the eye and the different quadrants of the conjunctiva relative to the limbus and cornea.

FIG. 4 illustrates injection of a steroid compound into the superotemporal quadrant of the subconjunctival space of the eye. The patient looks down while the thumb of one hand is used to gently retract the upper lid. The syringe containing the steroid-containing composition is placed tangential to the globe and inserted through the bulbar conjunctiva thereby introducing the needle into the subconjunctival space.

FIG. 5 shows a cross section of the eye and the location of the three zones of the conjunctiva (heavy black line)—palpebral, forniceal, and bulbar—relative to the anterior chamber and other anatomical regions in the eye.

### DETAILED DESCRIPTION

### Human Tear Lipocalin

The members of the lipocalin protein family (Pervaiz, S., and Brew, K. (1987) FASEB J. 1, 209-214) are typically small, secreted proteins which are characterized by a range of different molecular-recognition properties: their ability to bind various, principally hydrophobic molecules (such as retinoids, fatty acids, cholesterols, prostaglandins, biliverdins, pheromones, tastants, and odorants), their binding to specific cell-surface receptors, and their formation of macromolecular complexes.

The lipocalins share unusually low levels of overall sequence conservation, often with sequence identities of less than 20%. In strong contrast, their overall folding pattern is highly conserved. The central part of the lipocalin structure consists of a single eight-stranded anti-parallel β-sheet closed back on itself to form a continuously hydrogen-bonded β-barrel. One end of the barrel is sterically blocked by the N-terminal peptide segment that runs across its bottom as well as three peptide loops connecting the β-strands. The other end of the β-barrel is open to the solvent and encompasses a target-binding site, which is formed by four peptide loops.

Human tear pre-albumin, now called tear lipocalin (TLPC), was originally described as a major protein of human tear fluid (approximately one third of the total protein content) but has recently also been identified in several other secretory tissues including prostate, nasal mucosa and tracheal mucosa. Homologous proteins have been found in rat, pig, dog and horse. Tear lipocalin is an unusual lipocalin member because of its high promiscuity for relative insoluble lipids and binding characteristics that differ from other members of this protein family (reviewed in Redl, B. (2000) Biochim. Biophys. Acta 1482, 241-248). Tear lipocalin binds most strongly the least soluble lipids (Glasgow, B. J. et al. (1995) Curr. Eye Res. 14, 363-372; Gasymov, O. K. et al. (1999) Biochim. Biophys. Acta 1433, 307-320).

The method of the invention uses muteins of tear lipocalin that bind VEGF. “VEGF,” as used herein, means human vascular endothelial growth factor and all of its subtypes. Hence, a mutein of tear lipocalin that binds VEGF may bind one of human VEGF-A, VEGF-B, VEGF-C, or VEGF-D, or may bind a combination of any of the foregoing. “VEGF” may have the amino acid sequences set forth in SWISS PROT Data Bank Accession Nos. P15692, P49765, P49767, or 043915.

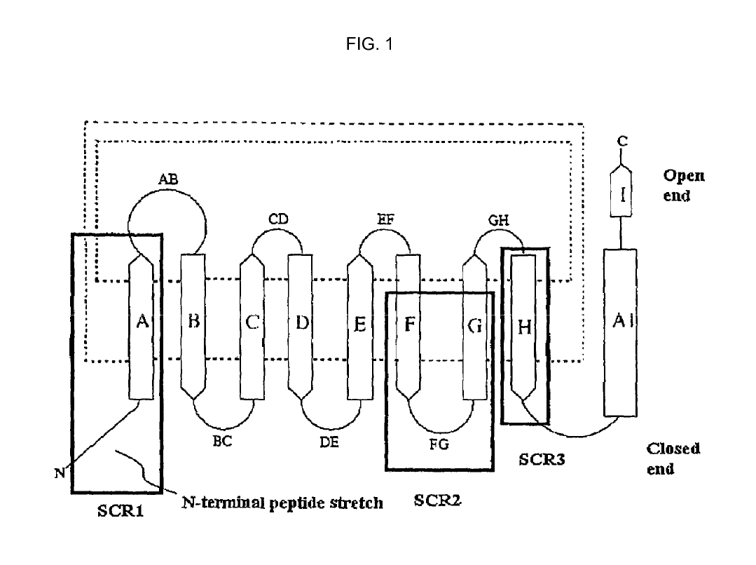

One can use in the method of the invention the muteins of tear lipocalin described in U.S. Pat. No. 7,585,940 and U.S. Pat. No. 7,893,208, both of the disclosures of which are incorporated herein by reference. Such lipocalin muteins are derived from a polypeptide of tear lipocalin or a homologue thereof, wherein the mutein comprises at least two mutated amino acid residues at any sequence position in the N-terminal peptide stretch and the three peptide loops BC, DE, and FG (cf. FIG. 1) arranged at the end of the β-barrel structure that is located opposite to the natural lipocalin binding pocket, wherein the tear lipocalin or homologue thereof has at least 60% sequence homology with human tear lipocalin, and wherein the mutein binds VEGF with detectable affinity.

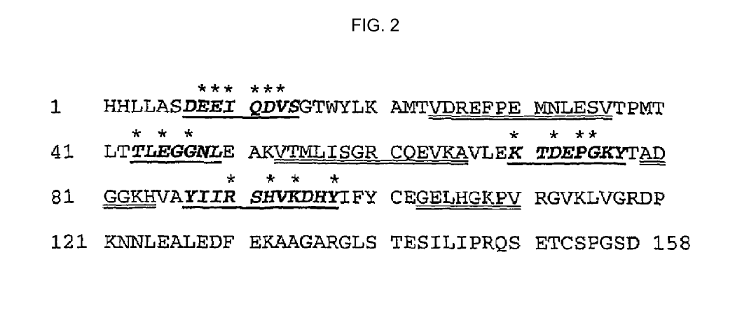

In this embodiment, amino acids in the three loops at the closed end of the internal ligand binding site of a tear lipocalin and/or the N-terminal peptide stretch of the tear lipocalin (cf. FIG. 2) can be mutated in order to obtain lipocalin muteins that bind VEGF with determinable affinity. Thus, this class of lipocalin muteins has antibody-like binding properties.

The mutein can also be derived from a polypeptide of tear lipocalin or a homologue thereof, wherein the mutein comprises at least two mutated amino acid residues at any sequence position in the four peptide loops AB, CD, EF, and GH (cf. FIG. 1) encompassing the natural lipocalin binding pocket, wherein the tear lipocalin or homologue thereof has at least 60% sequence homology with human tear lipocalin, and wherein the mutein binds VEGF with detectable affinity. Amino acids in the four loops at the open end of the ligand binding site of the lipocalins can be mutated for the generation of binding molecules against VEGF.

One can also use a mutein derived from a polypeptide of tear lipocalin or a homologue thereof, wherein the mutein comprises at least two mutated amino acid residues at any sequence position in the N-terminal region and the three peptide loops BC, DE, and FG arranged at the end of the β-barrel structure that is located opposite to the natural lipocalin binding pocket, wherein the mutein comprises at least two mutated amino acid residues at any sequence position in the four peptide loops AB, CD, EF, and GH encompassing the natural lipocalin binding pocket, wherein the tear lipocalin or homologue thereof has at least 60% sequence homology with human tear lipocalin, and wherein the mutein binds VEGF with detectable affinity. Thus, one can use a monomeric lipocalin mutein that due to the presence of two binding sites can have binding specifity for two given ligands. Such a bispecific molecule can be considered to be functionally equivalent to a bispecific antibody molecule such as a bispecific diabody. However, compared to a bispecific diabody (or antibody fragment in general), this class of bispecific lipocalin muteins has the advantage that it is composed only of one polypeptide chain whereas a diabody consists of two polypeptide chains that are non-covalently associated with each other. However such a bispecific mutein may also have only binding affinity for one given target.

The term “mutagenesis” as used herein means that the experimental conditions are chosen such that the amino acid naturally occurring at a given sequence position of the lipocalin used can be substituted by at least one amino acid that is not present at this specific position in the respective natural polypeptide sequence. The term “mutagenesis” also includes the (additional) modification of the length of sequence segments by deletion or insertion of one or more amino acids. Thus, it is within the scope of the invention that, for example, one amino acid at a chosen sequence position is replaced by a stretch of three random mutations, leading to an insertion of two amino acid residues compared to the length of (the respective segment) of the wild type protein. Such an insertion of deletion may be introduced independently from each other in any of the peptide segments that can be subjected to mutagenesis in the invention. In one exemplary embodiment of the invention, an insertion of several mutations is introduced in the loop AB of the selected lipocalin scaffold (cf. Examples 2 and 28, respectively). The term “random mutagenesis” means that no predetermined single amino acid (mutation) is present at a certain sequence position but that at least two amino acids can be incorporated into a selected sequence position during mutagenesis with a certain probability.

Such experimental conditions can, for example, be achieved by incorporating codons with a degenerate base composition into a nucleotide acid encoding the respective lipocalin employed. For example, use of the codon NNK or NNS (wherein N=adenine, guanine or cytosine or thymine; K=guanine or thymine; S=adenine or cytosine) allows incorporation of all 20 amino acids plus the amber stop codon during mutagenesis, whereas the codon WS limits the number of possibly incorporated amino acids to 12, since it excludes the amino acids Cys, Ile, Leu, Met, Phe, Trp, Tyr, Val from being incorporated into the selected position of the polypeptide sequence; use of the codon NMS (wherein M=adenine or cytosine), for example, restricts the number of possible amino acids to 11 at a selected sequence position since it excludes the amino acids Arg, Cys, Gly, Ile, Leu, Met, Phe, Trp, Val from being incorporated at a selected sequence position. In this respect it is noted that codons for other amino acids (than the regular 20 naturally occurring amino acids) such as selenocystein or pyrrolysine can also be incorporated into a nucleic acid of a mutein. It is also possible, as described by Wang, L., et al. (2001) Science 292, 498-500, or Wang, L., and Schultz, P. G. (2002) Chem. Commun. 1, 1-11, to use “artificial” codons such as UAG which are usually recognized as stop codons in order to insert other unusual amino acids, for example o-methyl-L-tyrosine or p-aminophenylalanine.

The term “tear lipocalin” as used herein is not limited to the human tear lipocalin (SWISS-PROT Data Bank Accession Number M90424) but is intended to include all polypeptides having the structurally conversed lipocalin fold as well as a sequence homology or a sequence identity with respect to the amino acid sequence of the human tear lipocalin of at least 60%. The term lipocalin fold is used in its regular meaning as used, e.g., in Flower, D. R. (1996), supra, to describe the typical three-dimensional lipocalin structure with a conformationally conserved β-barrel as a central motif made of a cylindrically closed β-sheet of eight antiparallel strands, wherein the open end of the barrel the β-strands are connected by four loops in a pairwise manner so that the binding pocket is formed (see also FIG. 1).

The definition of the peptide loops as used in the present invention is also in accordance with the regular meaning of the term lipocalin fold and is as follows and also illustrated in FIG. 1: The peptide loop (segment) AB connects the β-strands A and B of the cylindrically closed β-sheet, the peptide loop CD connects the β-strands C and D, the peptide loop EF connects the β-strands E and F, the peptide loop GH connects the β-strands G and H, the peptide loop BC connects the β-strands B and C, the loop DE connects the β-strands D and E, and the loop FG connects the β-strands F and G. As can be seen from FIG. 1 the loops AB, CD, EF and GH form the known binding site of the lipocalins (which was therefore called the open end), whereas, as found in the present invention, the loops BC, DE and FG can be used together with the N-terminal peptide stretch to form a second binding site which is located at the closed end of the β-barrel.

In accordance with the above, the term “tear lipocalin” includes structural homologues, already identified or yet to be isolated, from other species which have an amino acid sequence homology or sequence identity of more than about 60%. The term “homology” as used herein in its usual meaning and includes identical amino acids as well as amino acids which are regarded to be conservative substitutions (for example, exchange of a glutamate residue by a aspartate residue) at equivalent positions in the linear amino acid sequence of two proteins that are compared with each other. The term “sequence identity” or “identity” as used in the present invention means the percentage of pair-wise identical residues—following homology alignment of a sequence of a polypeptide of the present invention with a sequence in question—with respect to the number of residues in the longer of these two sequences.

The percentage of sequence homology or sequence identity is determined herein using the program BLASTP, version blastp 2.2.5 (Nov. 16, 2002; cf. Altschul, S. F. et al. (1997) Nucl. Acids Res. 25, 3389-3402). The percentage of homology is based on the alignment of the entire polypeptide sequences (matrix: BLOSUM 62; gap costs: 11.1; cutoff value set to 10³) including the propeptide sequences, using the human tear lipocalin as reference in a pairwise comparison. It is calculated as the percentage of numbers of “positives” (homologous amino acids) indicated as result in the BLASTP program output divided by the total number of amino acids selected by the program for the alignment. It is noted in this connection that this total number of selected amino acids can differ from the length of the tear lipocalin (176 amino acids including the propeptide) as it is seen in the following.

Examples of homologues proteins are Von Ebners gland protein 1 of Rattus norvegicus (VEGP protein; SWISS-PROT Data Bank Accession Numbers P20289) with a sequence homology of ca. 70% (125 positives/178 positions including the propeptide; when the 18 residues long propeptides containing 13 “positives” are not taken into account: 112 positives/160, resulting also in an homology of ca. 70%), Von Ebners gland protein 2 of Rattus norvegicus (VEG protein 2; SWISS-PROT Data Bank Accession Numbers P41244) with a sequence homology of ca. 71% (127 positives/178 including the propeptide; when the 18 residues long propeptides are not taken into account: 114 positives/160, the homology is determined to be also ca. 71%), Von Ebners gland protein 2 of Sus scrofra (pig) (LCN1; SWISS-PROT Data Bank Accession Numbers P53715) with a sequence homology of about 74% (131 positives/176 positions including the propeptide; when the 18 residues long propeptides containing 16 “positives” are not taken into account: 115 positives/158, resulting in an homology of ca. 73%), or the Major allergen Can f1 precursor of dog (ALL 1, SWISS-PROT Data Bank Accession Numbers 018873) with a sequence homology of ca. 70%, (122 positives/174 positions, or 110 positives/156=ca. 70% homology, when the propeptides with 12 positives are excluded) as determined with the program BLASTP as explained above. Such a structural homologue of the tear lipocalin can be derived from any species, i.e. from prokaryotic as well as from eukaryotic organisms. In case of eukaryotic organisms, the structural homologue can be derived from invertebrates as well as vertebrates such as mammals (e.g., human, monkey, dog, rat or mouse) or birds or reptiles.

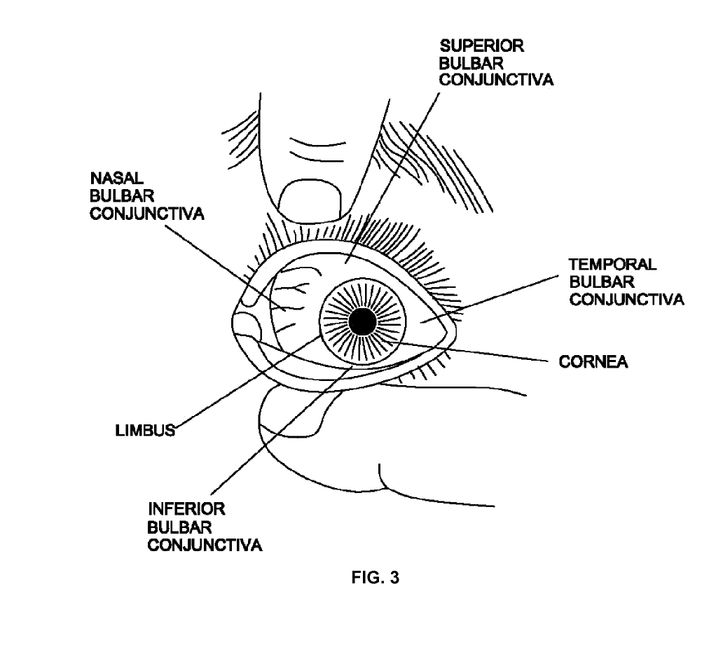

In case a protein other than tear lipocalin is used in the present invention, the definition of the mutated sequence positions given for tear lipocalin can be assigned to the other lipocalin with the help of published sequence alignments or alignments methods which are available to the skilled artisan. A sequence alignment can, for example, be carried out as explained in WO 99/16873 (cf. FIG. 3 therein), using an published alignment such as the one in FIG. 1 of Redl, B. (2000) Biochim. Biophys. Acta 1482, 241-248. If the three-dimensional structure of the lipocalins are available structural superpositions can also be used for the determination of those sequence positions that are to be subjected to mutagenesis in the present invention. Other methods of structural analysis such as multidimensional nuclear magnetic resonance spectroscopy can also be employed for this purpose.

The homologue of tear lipocalin can also be a mutein protein of tear lipocalin itself, in which amino acid substitutions are introduced at positions other than the positions selected in the present invention. For example, such a mutein can be a protein in which positions at the solvent exposed surface of the β-barrel are mutated compared to the wild type sequence of the tear lipocalin in order to increase the solubility or the stability of the protein.

In general, the term “tear lipocalin” includes all proteins that have a sequence homology or sequence identity of more than 60%, 70% 80%, 85%, 90%, or 95% in relation to the human tear lipocalin (SWISS-PROT Data Bank Accession Number M90424).

In one embodiment the mutein is derived from human tear lipocalin. In other embodiments the mutein is derived from the VEGP protein, VEG protein 2, LCN 1, or ALL 1 protein.

If the binding site at the closed end of the β-barrel is used, the mutein according to the invention typically comprises mutations at any two or more of the sequence positions in the peptide segments corresponding to the sequence positions 7-14, 41-49, 69-77, and 87-98 of the linear polypeptide sequence of human tear lipocalin. The positions 7-14 are part of the N-terminal peptide stretch, the positions 41-49 are comprised in the BC loop, the positions 60-77 are comprised in the DE loop and the positions 87-98 are comprised in the FG loop.

In more specific embodiments of those muteins the mutations are introduced at those sequence positions, which correspond to the positions 8, 9, 10, 11, 12, 13, 43, 45, 47, 70, 72, 74, 75, 90, 92, 94, and 97 of human tear lipocalin. Usually, such a mutein comprises mutations at 5-10 or 12-16 or all 17 of the sequence positions.

In case the binding site at the open end of the β-barrel is subjected to mutagenesis a lipocalin mutein according to the invention comprises mutations at any two or more of the sequence positions in the peptide segments corresponding to the sequence positions 24-36, 53-66, 79-84, and 103-110 of the linear polypeptide sequence of human tear lipocalin. The positions 24-36 are comprised in the AB loop, the positions 53-66 are comprised in the CD loop, the positions 69-77 are comprised in the EF loop and the positions 103-110 are comprised in the GH loop. In one embodiment of the invention, an insertion of 1 to 6 amino acid residues, preferably of 2 to 4 amino acid residues, is introduced into the peptide segment hat is formed by the sequence positions corresponding to sequence positions 24-36 of human tear lipocalin. This insertion can be included at any position within this segment. In one exemplary embodiment, this insertion is introduced between sequence positions 24 and 25 of human tear lipocalin. However, it is also noted again that the introduction of a stretch of at least two amino acids into a peptide segment that is part of the binding sites used here, is not limited to the segment comprising residues 24-26 but can be included in any segment participating in the formation of one of the two binding sites chosen herein.

Accordingly, a mutein having two binding sites comprises mutations at any two or more of the sequence positions in the peptide segments corresponding to the sequence positions 7-14, 41-49, 69-77, and 87-97 of the linear polypeptide sequence of human tear lipocalin and additional mutations at any two or more of the sequence positions in the peptide segments corresponding to the sequence positions 24-36, 53-66, 79-84, and 103-110 of the linear polypeptide sequence of human tear lipocalin.

In this respect it is noted that the number of the segments (loops) defined above which are used for mutagenesis can vary (the N-terminal peptide stretch is included in the meaning of the term segment or loop). It is not necessary to mutate all four of these segments all together of each of the two binding sites, for example in a concerted mutagenesis. But it is also possible to introduce mutations only in one, two or three segments of each binding site in order to generate a mutein having detectable affinity to a given target. Therefore, it is possible to subject, for example, only two or three segments at the closed end of the β-barrel to mutagenesis if a binding molecule with only one engineered binding site is wanted. If this molecule is then wanted to have binding affinity towards a second target, sequence positions in any of the four loops of the second binding site can then be mutated. It is also possible, however, to mutate peptide loops of both binding sites, even if a given target is to be bound by one of the binding site only.

The lipocalin muteins of the invention may comprise the wild type (natural) amino acid sequence outside the mutated segments. On the other hand, the lipocalin muteins disclosed herein may also contain amino acid mutations outside the sequence positions subjected to mutagenesis as long as those mutations do not interfere with the binding activity and the folding of the mutein. Such mutations can be accomplished very easily on DNA level using established standard methods (Sambrook, J. et al. (1989) Molecular Cloning: A Laboratory Manual, 2nd Ed., Cold Spring Harbor Laboratory Press, Cold Spring Harbor, N.Y.). Possible alterations of the amino acid sequence are insertions or deletions as well as amino acid substitutions. Such substitutions may be conservative, i.e. an amino acid residue is replaced with a chemically similar amino acid residue. Examples of conservative substitutions are the replacements among the members of the following groups: 1) alanine, serine, and threonine; 2) aspartic acid and glutamic acid; 3) 11dentate11es and glutamine; 4) arginine and lysine; 5) isoleucine, leucine, methionine, and valine; and 6) phenylalanine, tyrosine, and tryptophan. One the other hand, it is also possible to introduce non-conservative alterations in the amino acid sequence.

Such modifications of the amino acid sequence include directed mutagenesis of single amino acid positions in order to simplify sub-cloning of the mutated lipocalin gene or its parts by incorporating cleavage sites for certain restriction enzymes. In addition, these mutations can also be incorporated to further improve the affinity of a lipocalin mutein for a given target (cf. Examples 17-19 and 24). In one embodiment, a mutation is introduced in at least one of the sequence positions (of the lipocalin framework) that correspond to sequence positions 21, 50, 51 and 83 of the linear polypeptide sequence of human tear lipocalin. Furthermore, mutations can be introduced in order to modulate certain characteristics of the mutein such as to improve folding stability or water solubility or to reduce aggregation tendency, if necessary.

The lipocalin muteins of the invention are able to bind the desired target with detectable affinity, i.e. with an affinity constant of preferably at least 10⁵ M⁻¹. Lower affinities are generally no longer measurable with common methods such as ELISA and therefore of secondary importance. Especially preferred are lipocalin muteins, which bind the desired target with an affinity of at least 10⁶ M⁻¹, corresponding to a dissociation constant of the complex of 1 μM. The binding affinity of a mutein to the desired target can be measured by a multitude of methods such as fluorescence titration, competition ELISA or surface 11dentat resonance.

It is clear to the skilled person that complex formation is dependent on many factors such as concentration of the binding partners, the presence of competitors, ionic strength of the buffer system etc. Selection and enrichment is generally performed under conditions allowing the isolation of lipocalin muteins having an affinity constant of at least 10⁵ M⁻¹ to the target.

However, the washing and elution steps can be carried out under varying stringency. A selection with respect to the kinetic characteristics is possible as well. For example, the selection can be performed under conditions, which favor complex formation of the target with muteins that show a slow dissociation from the target, or in other words a low k_{off} rate.

A tear lipocalin mutein of the invention typically exists as monomeric protein. However, it is also possible that an inventive lipocalin mutein is able to spontaneously dimerise or oligomerise. Although the use of lipocalin muteins that form stable monomers is usually preferred due to the simplified handling of the protein, for example, the use of lipocalin muteins that form stable homodimers or multimers can even be preferred here since such multimers can provide for a (further) increased affinity and/or avidity to a given target. Furthermore, oligomeric forms of the lipocalin mutein may have prolonged serum half-life.

For some applications, it is useful to employ the muteins of the invention in a labeled form. Accordingly, the invention is also directed to lipocalin muteins which are conjugated to a label selected from the group consisting of enzyme labels, radioactive labels, colored labels, fluorescent labels, chromogenic labels, luminescent labels, haptens, digoxigenin, biotin, metal complexes, metals, and colloidal gold. The mutein may also be conjugated to an organic molecule. The term “organic molecule” as used herein preferably denotes an organic molecule comprising at least two carbon atoms, but preferably not more than seven rotatable carbon bonds, having a molecular weight in the range between 100 and 2000 Dalton, preferably 1000 Dalton, and optionally including one or two metal atoms.

In general, it is possible to label the lipocalin mutein with any appropriate chemical substance or enzyme, which directly or indirectly generates a detectable compound or signal in a chemical, physical or enzymatic reaction. An example for a physical reaction is the emission of fluorescence upon irradiation or the emission of X-rays when using a radioactive label. Alkaline phosphatase, horseradish peroxidase or β-galactosidase are examples of enzyme labels which catalyze the formation of chromogenic reaction products. In general, all labels commonly used for antibodies (except those exclusively used with the sugar moiety in the Fc part of immunoglobulins) can also be used for conjugation to the muteins of the present invention. The muteins of the invention may also be conjugated with any suitable therapeutically active agent, e.g., for the targeted delivery of such agents to a given cell, tissue or organ or for the selective targeting of cells, e.g., of tumor cells without affecting the surrounding normal cells. Examples of such therapeutically active agents include radionuclides, toxins, small organic molecules, and therapeutic peptides (such as peptides acting as agonists/antagonists of a cell surface receptor or peptides competing for a protein binding site on a given cellular target). The lipocalin muteins of the invention may, however, also be conjugated with therapeutically active nucleic acids such as antisense nucleic acid molecules, small interfering RNAs, micro RNAs or ribozymes. Such conjugates can be produced by methods well known in the art.

For several applications of the muteins disclosed herein it may be advantageous to use them in the form of fusion proteins. In preferred embodiments, the inventive lipocalin mutein is fused at its N-terminus or its C-terminus to a protein, a protein domain or a peptide such as a signal sequence and/or an affinity tag.

The fusion partner may confer new characteristics to the inventive lipocalin mutein such as enzymatic activity or binding affinity for other molecules. Examples of suitable fusion proteins are alkaline phosphatase, horseradish peroxidase, gluthation-S-transferase, the albumin-binding domain of protein G, protein A, antibody fragments, oligomerization domains, lipocalin muteins of same or different binding specificity (which results in the formation of “duocalins”, cf. Schlehuber, S., and Skerra, A. (2001), Biol. Chem. 382, 1335-1342), or toxins. In particular, it may be possible to fuse a lipocalin mutein of the invention with a separate enzyme active site such that both “components” of the resulting fusion protein together act on a given therapeutic target. The binding domain of the lipocalin mutein attaches to the disease-causing target, allowing the enzyme domain to abolish the biological function of the target. If two bispecific lipocalin muteins of the inventions (i.e. each of them has two binding sites) are combined into a “duocalin”, a tetravalent molecule is formed. If for example a duocalin is generated from only one mutein having two binding sites that specifically bind biotin, a tetravalent molecule (homodimer) comparable to streptavidin (which is a homotetramer, in which each monomer binds one biotin molecule) can be obtained. Due to expected avidity effects such a mutein might be a useful analytical tool in methods that make use of the detection of biotin groups. A lipocalin mutein that spontaneously forms homodimers or—multimers can, of course, also be used for such a purpose.

Affinity tags such as the STREP-TAG® or STREP-TAG® II (strepavidin tag used for detection or purification of recombinant proteins) (Schmidt, T. G. M. et al. (1996) J. Mol. Biol. 255, 753-766), the myc-tag, the FLAG-tag, the His₆-tag or the HA-tag or proteins such as glutathione-S-transferase also allow easy detection and/or purification of recombinant proteins are further examples of preferred fusion partners. Finally, proteins with chromogenic or fluorescent properties such as the green fluorescent protein (GFP) or the yellow fluorescent protein (YFP) are suitable fusion partners for a lipocalin mutein of the invention as well.

The term “fusion protein” as used herein also comprises lipocalin muteins according to the invention containing a signal sequence. Signal sequences at the N-terminus of a polypeptide direct this polypeptide to a specific cellular compartment, for example the periplasm of E. coli or the endoplasmatic reticulum of eukaryotic cells. A large number of signal sequences is known in the art. A preferred signal sequence for secretion a polypeptide into the periplasm of E. coli is the OmpA-signal sequence.

The present invention also relates to nucleic acid molecules (DNA and RNA) comprising nucleotide sequences coding for muteins as described herein. Since the degeneracy of the genetic code permits substitutions of certain codons by other codons specifying the same amino acid, the invention is not limited to a specific nucleic acid molecule encoding a fusion protein of the invention but includes all nucleic acid molecules comprising nucleotide sequences encoding a functional fusion protein.

In one preferred embodiment of the nucleic acid molecule of invention its sequence is derived from the coding sequence of human tear lipocalin. In other preferred embodiments the nucleic acid is derived from the VEGP protein, VEG protein 2, LCN 1 or ALL 1 protein

In another preferred embodiment the nucleic acid sequence encoding a mutein according to the invention comprises mutations at any two or more of the sequence positions in the peptide segments corresponding to the sequence positions 7-14, 43-49, 70-77, and 87-97 of the linear polypeptide sequence of human tear lipocalin with the sequence positions corresponding to the positions 8, 9, 10, 11, 12, 13, 43, 45, 47, 70, 72, 74, 75, 90, 92, 94, and 97 of human tear lipocalin being particularly preferred.

In a further preferred embodiment the nucleic acid sequence encoding a mutein according to the invention comprises mutations at any two or more of the sequence positions in the peptide segments corresponding to the sequence positions 24-36, 53-66, 79-84, and 103-110 of the linear polypeptide sequence of human tear lipocalin.

Also preferred are nucleic acid molecules encoding a mutein of the invention comprising mutations at any two or more of the sequence positions in the peptide segments corresponding to the sequence positions 7-14, 43-49, 70-77, and 87-97 of the linear polypeptide sequence of human tear lipocalin mutations and additional mutations at any two or more of the sequence positions in the peptide segments corresponding to the sequence positions 24-36, 53-66, 79-84, and 103-110 of the linear polypeptide sequence of human tear lipocalin.

The invention as disclosed herein also includes nucleic acid molecules encoding TLPC muteins, which comprise additional mutations outside the segments of experimental mutagenesis. Such mutations are often tolerated or can even prove to be advantageous, for example if they contribute to an improved folding efficiency, protein stability or ligand binding affinity of the mutein.

A nucleic acid molecule disclosed in this application may be “operably linked” to a regulatory sequence (or regulatory sequences) to allow expression of this nucleic acid molecule.

A nucleic acid molecule, such as DNA, is referred to as “capable of expressing a nucleic acid molecule” or capable “to allow expression of a nucleotide sequence” if it comprises sequence elements which contain information regarding to transcriptional and/or translational regulation, and such sequences are “operably linked” to the nucleotide sequence encoding the polypeptide. An operable linkage is a linkage in which the regulatory sequence elements and the sequence to be expressed are connected in a way that enables gene expression. The precise nature of the regulatory regions necessary for gene expression may vary among species, but in general these regions comprise a promoter which, in prokaryotes, contains both the promoter per se, i.e. DNA elements directing the initiation of transcription, as well as DNA elements which, when transcribed into RNA, will signal the initiation of translation. Such promoter regions normally include 5′non-coding sequences involved in initiation of transcription and translation, such as the −35/−10 boxes and the Shine-Dalgarno element in prokaryotes or the TATA box, CAAT sequences, and 5′-capping elements in eukaryotes. These regions can also include enhancer or repressor elements as well as translated signal and leader sequences for targeting the native polypeptide to a specific compartment of a host cell.

In addition, the 3′ non-coding sequences may contain regulatory elements involved in transcriptional termination, polyadenylation or the like. If, however, these termination sequences are not satisfactory functional in a particular host cell, then they may be substituted with signals functional in that cell.

Therefore, a nucleic acid molecule of the invention can include a regulatory sequence, preferably a promoter sequence. In another preferred embodiment, a nucleic acid molecule of the invention comprises a promoter sequence and a transcriptional termination sequence. Suitable prokaryotic promoters are, for example, the tet promoter, the lacUV5 promoter or the T7 promoter. Examples of promoters useful for expression in eukaryotic cells are the SV40 promoter or the CMV promoter.

The nucleic acid molecules of the invention can also be comprised in a vector or any other cloning vehicles, such as plasmids, phagemids, phage, baculovirus, cosmids or artificial chromosomes. In a preferred embodiment, the nucleic acid molecule is comprised in a phasmid. A phasmid vector denotes a vector encoding the intergenic region of a temperent phage, such as M13 or f1, or a functional part thereof fused to the cDNA of interest. After superinfection of the bacterial host cells with such an phagemid vector and an appropriate helper phage (e.g. M13K07, VCS-M13 or R408) intact phage particles are produced, thereby enabling physical coupling of the encoded heterologous cDNA to its corresponding polypeptide displayed on the phage surface (reviewed, e.g., in Kay, B. K. et al. (1996) Phage Display of Peptides and Proteins—A Laboratory Manual, 1st Ed., Academic Press, New York N.Y.; Lowman, H. B. (1997) Annu. Rev. Biophys. Biomol. Struct. 26, 401-424, or Rodi, D. J., and Makowski, L. (1999) Curr. Opin. Biotechnol. 10, 87-93).

Such cloning vehicles can include, aside from the regulatory sequences described above and a nucleic acid sequence encoding a lipocalin mutein of the invention, replication and control sequences derived from a species compatible with the host cell that is used for expression as well as selection markers conferring a selectable phenotype on transformed or transfected cells. Large numbers of suitable cloning vectors are known in the art, and are commercially available.

The DNA molecule encoding lipocalin muteins of the invention, and in particular a cloning vector containing the coding sequence of such a lipocalin mutein can be transformed into a host cell capable of expressing the gene. Transformation can be performed using standard techniques (Sambrook, J. et al. (1989), supra). Thus, the invention is also directed to a host cell containing a nucleic acid molecule as disclosed herein.

The transformed host cells are cultured under conditions suitable for expression of the nucleotide sequence encoding a fusion protein of the invention. Suitable host cells can be prokaryotic, such as Escherichia coli (E. coli) or Bacillus subtilis, or eukaryotic, such as Saccharomyces cerevisiae, Pichia pastoris, SF9 or High5 insect cells, immortalized mammalian cell lines (e.g. HeLa cells or CHO cells) or primary mammalian cells.

The invention also relates to a method for the generation of a mutein according to the invention or a fusion protein thereof, comprising: (a) subjecting a nucleic acid molecule encoding a tear lipocalin or a homologue thereof, wherein the tear lipocalin or homologue thereof has at least 60% sequence homology with human tear lipocalin, to mutagenesis at two or more different codons, resulting in one or more mutein nucleic acid molecules(s); (b) expressing the one or more mutein nucleic acid molecule(s) obtained in (a) in a suitable expression system, and (c) enriching at least one mutein having a detectable binding affinity for a given target by means of selection and/or isolation.

In further embodiments of this method, the nucleic acid molecule can be individually subjected to mutagenesis at two or more different codons (i.e., usually nucleotide triplets) in any one, two, three or all four above-mentioned peptide segments arranged at either end of the β-barrel structure. Accordingly, it is sufficient to exchange only one base in a codon if this exchange results in a change of the encoded amino acid.

In the method of generation a mutein or a fusion protein thereof is obtained starting from the nucleic acid encoding tear lipocalin or a homologue thereof, which is subjected to mutagenesis and introduced into a suitable bacterial or eukaryotic host organism by means of recombinant DNA technology (as already outlined above).

The coding sequence of, for example, human tear lipocalin (Redl, B. et al. (1992) J. Biol. Chem. 267, 20282-20287) can serve as a starting point for mutagenesis of the peptide segments selected in the present invention. For the mutagenesis of the amino acids in the N-terminal peptide stretch and the three peptide loops BC, DE, and FG at the end of the β-barrel structure that is located opposite to the natural lipocalin binding pocket as well as the four peptide loops AB, CD, EF, and GH encompassing the binding pocket, the person skilled in the art has at his disposal the various established standard methods for site-directed mutagenesis (Sambrook, J. et al. (1989), supra). A commonly used technique is the introduction of mutations by means of PCR (polymerase chain reaction) using mixtures of synthetic oligonucleotides, which bear a degenerate base composition at the desired sequence positions. The use of nucleotide building blocks with reduced base pair specificity, as for example inosine, is another option for the introduction of mutations into a chosen sequence segment. A further possibility is the so-called triplet-mutagenesis. This method uses mixtures of different nucleotide triplets each of which codes for one amino acid for the incorporation into the coding sequence.

One possible strategy for introducing mutations in the selected regions of the respective polypeptides is based on the use of four oligonucleotides, each of which is partially derived from one of the corresponding sequence segments to be mutated. When synthesizing these oligonucleotides, a person skilled in the art can employ mixtures of nucleic acid building blocks for the synthesis of those nucleotide triplets which correspond to the amino acid positions to be mutated so that codons encoding all natural amino acids randomly arise, which at last results in the generation of a lipocalin peptide library. For example, the first oligonucleotide corresponds in its sequence—apart from the mutated positions—to the coding strand for the peptide segment to be mutated at the most N-terminal position of the lipocalin polypeptide. Accordingly, the second oligonucleotide corresponds to the non-coding strand for the second sequence segment following in the polypeptide sequence. The third oligonucleotide corresponds in turn to the coding strand for the corresponding third sequence segment. Finally, the fourth oligonucleotide corresponds to the non-coding strand for the fourth sequence segment. A polymerase chain reaction can be performed with the respective first and second oligonucleotide and separately, if necessary, with the respective third and fourth oligonucleotide.

The amplification products of both of these reactions can be combined by various known methods into a single nucleic acid comprising the sequence from the first to the fourth sequence segments, in which mutations have been introduced at the selected positions. To this end, both of the products can for example be subjected to a new polymerase chain reaction using flanking oligonucleotides as well as one or more mediator nucleic acid molecules, which contribute the sequence between the second and the third sequence segment. In the choice of the number and arrangement within the sequence of the oligonucleotides used for the mutagenesis, the person skilled in the art has numerous alternatives at his disposal.

The nucleic acid molecules defined above can be connected by ligation with the missing 5′- and 3′-sequences of a nucleic acid encoding a lipocalin polypeptide and/or the vector, and can be cloned in a known host organism. A multitude of established procedures are available for ligation and cloning (Sambrook, J. et al. (1989), supra). For example, recognition sequences for restriction endonucleases also present in the sequence of the cloning vector can be engineered into the sequence of the synthetic oligonucleotides. Thus, after amplification of the respective PCR product and enzymatic cleavage the resulting fragment can be easily cloned using the corresponding recognition sequences.

Longer sequence segments within the gene coding for the protein selected for mutagenesis can also be subjected to random mutagenesis via known methods, for example by use of the polymerase chain reaction under conditions of increased error rate, by chemical mutagenesis or by using bacterial mutator strains. Such methods can also be used for further optimization of the target affinity or specificity of a lipocalin mutein. Mutations possibly occurring outside the segments of experimental mutagenesis are often tolerated or can even prove to be advantageous, for example if they contribute to an improved folding efficiency or folding stability of the lipocalin mutein.

After expression of the nucleic acid sequences that were subjected to mutagenesis in an appropriate host, the clones carrying the genetic information for the plurality of respective lipocalin muteins, which bind a given target can be selected from the library obtained. Well known techniques can be employed for the selection of these clones, such as phage display (reviewed in Kay, B. K. et al. (1996) supra; Lowman, H. B. (1997) supra or Rodi, D. J., and Makowski, L. (1999) supra), colony screening (reviewed in Pini, A. et al. (2002) Comb. Chem. High Throughput Screen. 5, 503-510), ribosome display (reviewed in Amstutz, P. et al. (2001) Curr. Opin. Biotechnol. 12, 400-405) or mRNA display as reported in Wilson, D. S. et al. (2001) Proc. Natl. Acad. Sci. USA 98, 3750-3755.

An embodiment of the phage display technique (reviewed in Kay, B. K. et al. (1996), supra; Lowman, H. B. (1997) supra or Rodi, D. J., and Makowski, L. (1999), supra) using temperent M13 phage is given as an example of a selection method according to the invention. However, it is noted that other temperent phage such as f1 or lytic phage such as T7 may be employed as well. For the exemplary selection method, M13 phagemids (cf. also above) are produced which allow the expression of the mutated lipocalin nucleic acid sequence as a fusion protein with a signal sequence at the N-terminus, preferably the OmpA-signal sequence, and with the capsid protein pIII of the phage M13 or fragments thereof capable of being incorporated into the phage capsid at the C-terminus. The C-terminal fragment .DELTA.pIII of the phage capsid protein comprising amino acids 217 to 406 of the wild type sequence is preferably used to produce the fusion proteins. Especially preferred is a C-terminal fragment of pIII, in which the cysteine residue at position 201 is missing or is replaced by another amino acid.

The fusion protein may comprise additional components such as an affinity tag, which allows the immobilization and/or purification of the fusion protein or its parts. Furthermore, a stop codon can be located between the sequence regions encoding the lipocalin or its muteins and the phage capsid gene or fragments thereof, wherein the stop codon, preferably an amber stop codon, is at least partially translated into an amino acid during translation in a suitable suppressor strain.

For example, the phagemid vector pTLPC7 can be used for the construction of a phage library encoding human tear lipocalin muteins. The inventive nucleic acid molecules coding for the mutated peptide segments are inserted into the vector using the BstXI restriction sites. Recombinant vectors are then transformed into a suitable host strain such as E. coli XL1-Blue. The resulting library is subsequently superinfected in liquid culture with an appropriate M13-helper phage in order to produce functional phage. The recombinant phagemid displays the lipocalin mutein on its surface as a fusion with the coat protein pIII or a fragment thereof, while the N-terminal signal sequence of the fusion protein is normally cleaved off. On the other hand, it also bears one or more copies of the native capsid protein pIII supplied by the helper phage and is thus capable of infecting a recipient, in general a bacterial strain carrying a F- or F′-plasmid. During or after infection gene expression of the fusion protein comprised of the lipocalin mutein and the capsid protein pIII can be induced, for example by addition of anhydrotetracycline. The induction conditions are chosen such that a substantial fraction of the phage obtained displays at least one lipocalin mutein on their surface. Various methods are known for isolating the phage, such as precipitation with polyethylene glycol. Isolation typically occurs after an incubation period of 6-8 hours.

The isolated phage are then subjected to a selection process by incubating them with a given target, wherein the target is present in a form allowing at least a temporary immobilization of those phage displaying muteins with the desired binding activity. Several immobilization methods are known in the art. For example, the target can be conjugated with a carrier protein such as serum albumin and be bound via this carrier to a protein-binding surface such as polystyrene. Microtiter plates suitable for ELISA techniques or so-called “immunosticks” are preferred. Alternatively, conjugates of the target can also be implemented with other binding groups such as biotin. The target can then be immobilized on surfaces, which will selectively bind this group, such as microtiter plates or paramagnetic particles coated with avidin or streptavidin.

For example, the phage particles are captured by binding to the respective target immobilized on the surface. Unbound phage particles are subsequently removed by iterative washing. For the elution of bound phage, free target (ligand) molecules can be added to the samples as a competitor. Alternatively, elution can also be achieved by adding proteases or under moderately denaturing conditions, e.g. in the presence of acids, bases, detergents or chaotropic salts. A preferred method is the elution using buffers having pH 2.2, followed by neutralization of the solution. The eluted phage may then be subjected to another selection cycle. Preferably, selection is continued until at least 0.1% of the clones comprise lipocalin muteins with detectable affinity for the respective target. Depending on the complexity of the library employed 2-8 cycles are required to this end.

For the functional analysis of the selected lipocalin muteins, an E. coli host strain is infected with the phagemids obtained and phagemid DNA is isolated using standard techniques (Sambrook, J. et al. (1989), supra). The mutated sequence fragment or the entire lipocalin mutein nucleic acid sequence can be sub-cloned in any suitable expression vector. The recombinant lipocalin muteins obtained can be purified from their host organism or from a cell lysate by various methods known in the art such as gel filtration or affinity chromatography.

However, the selection of lipocalin muteins can also be performed using other methods well known in the art. Furthermore, it is possible to combine different procedures. For example, clones selected or at least enriched by phage display can subsequently be subjected to a colony-screening assay in order to directly isolate a particular lipocalin mutein with detectable binding affinity for a given target. Additionally, instead of generating a single phage library comparable methods can be applied in order to optimize a mutein with respect to its affinity or specificity for the desired target by repeated, optionally limited mutagenesis of its coding nucleic acid sequence.

Once a mutein with affinity to a given target have been selected, it is additionally possible to subject such a mutein to further mutagenesis in order to select variants of even higher affinity from the new library thus obtained. The affinity 22dentate22e can be achieved by site specific mutation based on rational design or a random mutation One possible approach for affinity maturation is the use of error-prone PCR, which results in point mutations over a selected range of sequence positions of the lipocalin mutein (cf. Example 17). The error prone PCR can be carried out in accordance with any known protocol such as the one described by Zaccolo et al. (1996) J. Mol. Biol. 255, 589-603. Other methods of random mutagenesis that are suitable for affinity maturation include random insertion/deletion (RID) mutagenesis as described by Murakami, H et al. (2002) Nat. Biotechnol. 20, 76-81 or nonhomologous random recombination (NRR) as described by Bittker, J. A et al. (2002) Nat. Biotechnol. 20, 1024-1029. Affinity maturation can also be carried out according to the procedure described in WO 00/75308 or Schlehuber, S. et al. (2000) J. Mol. Biol. 297, 1105-1120, where muteins of the bilin-binding protein having high affinity to digoxigenin were obtained.

The invention also relates to a method for the production of a mutein of the invention, wherein the mutein, a fragment of the mutein or a fusion protein of the mutein and another polypeptide is produced starting from the nucleic acid coding for the mutein by means of genetic engineering methods. The method can be carried out in vivo, the mutein can for example be produced in a bacterial or 23dentate23e host organism and then isolated from this host organism or its culture. It is also possible to produce a protein in vitro, for example by use of an in vitro translation system.

When producing the mutein in vivo a nucleic acid encoding a mutein of the invention is introduced into a suitable bacterial or eukaryotic host organism by means of recombinant DNA technology (as already outlined above). For this purpose, the host cell is first transformed with a cloning vector comprising a nucleic acid molecule encoding a mutein of the invention using established standard methods (Sambrook, J. et al. (1989), supra). The host cell is then cultured under conditions, which allow expression of the heterologous DNA and thus the synthesis of the corresponding polypeptide. Subsequently, the polypeptide is recovered either from the cell or from the cultivation medium. Since many lipocalins comprise intramolecular disulfide bonds, it can be preferred to direct the polypeptide to a cell compartment having an oxidizing redox-milieu using an appropriate signal sequence. Such an oxidizing environment is provided in the periplasm of Gram-negative bacteria such as E. coli or in the lumen of the endoplasmatic reticulum of eukaryotic cells and usually favors the correct formation of the disulfide bonds. It is, however, also possible to generate a mutein of the invention in the cytosol of a host cell, preferably E. coli. In this case, the polypeptide can, for instance, be produced in form of inclusion bodies, followed by renaturation in vitro. A further option is the use of specific host strains having an oxidizing intracellular milieu, which thus allow the production of the native protein in the cytosol.

However, a mutein of the invention may not necessarily be generated or produced only by use of genetic engineering. Rather, a lipocalin mutein can also be obtained by chemical synthesis such as Merrifield solid phase polypeptide synthesis. It is for example possible that promising mutations are identified using molecular modeling and then to synthesize the wanted (designed) polypeptide in vitro and investigate the binding activity for a given target. Methods for the solid phase and/or solution phase synthesis of proteins are well known in the art (reviewed, e.g., in Lloyd-Williams, P. et al. (1997) Chemical Approaches to the Synthesis of Peptides and Proteins. CRC Press, Boca Raton, Fields, G. B., and Colowick, S. P. (1997) Solid-Phase Peptide Synthesis. Academic Press, San Diego, or Bruckdorfer, T. et al. (2004) Curr. Pharm. Biotechnol. 5, 29-43).

In another embodiment, one can use in the method of the invention the muteins of tear lipocalin described in each of U.S. Pat. No. 7,250,297 (WO 00/75308), U.S. Pat. No. 7,001,882 (WO 99/16873), U.S. Pat. No. 7,252,998 (WO 03/029463), and U.S. Pat. No. 7,118,915 (WO 03/029471), and U.S. Patent Application Publication No. 2009/0305982, all of the disclosures of which are incorporated by reference herein, provided that such muteins of tear lipocalin bind to VEGF. WO 99/16873 discloses the class of Anticalins®, that is, polypeptides of the lipocalin family with mutated amino acid positions in the region of the four peptide loops, which are arranged at the end of the cylindrical β-barrel structure encompassing the binding pocket, and which correspond to those segments in the linear polypeptide sequence comprising the amino acid positions 28 to 45, 58 to 69, 86 to 99, and 114 to 129 of the bilin-binding protein of Pieris brassicae. WO 00/75308 discloses muteins of the bilin-binding protein, which specifically bind digoxigenin, whereas WO 03/029463 and WO 03/029471 relate to muteins of the human 24dentate24es gelatinase-associated lipocalin and apolipoprotein D, respectively.

### Methods of Treatment

The lipocalin muteins described herein are administered to the eye to treat neovascular diseases of the anterior segment of the eye. To “treat,” as used here, means to deal with medically, and includes, for example, administering lipocalin muteins to reduce the severity of disease, to ameliorate its symptoms, and to prevent its occurrence.

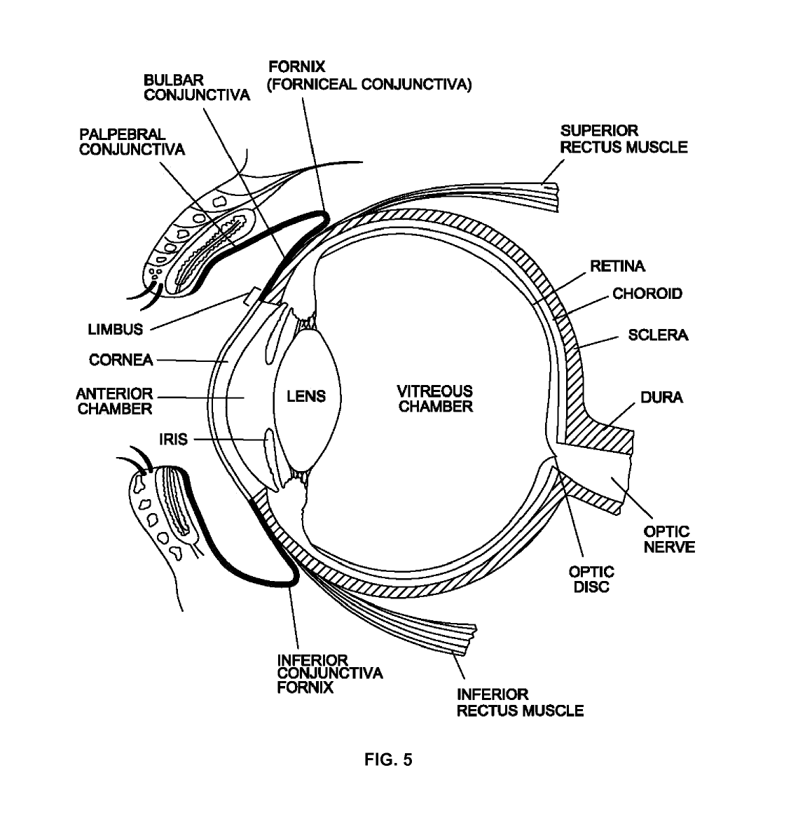

In one embodiment, the lipocalin muteins are topically to the eye. In another embodiment, the lipocalin muteins are administered to the subconjunctival space (FIGS. 3-5) or the anterior chamber (FIG. 5).

As used herein, the “subconjunctival space” refers to any of the following: (1) the potential space between the bulbar conjunctiva and Tenon's capsule and extending from the limbus to the fornix; (2) the potential space between the palpebral conjunctiva and the tarsus and extending from the eye lid margin (mucocutaneous junction of the eyelid) to the fornix; and (3) the potential space just beneath the forniceal conjunctiva at the junctional bay or fornix. The subconjunctival space is therefore the potential space just beneath the conjunctiva from the limbus, around the fornix, to the eye lid margin.

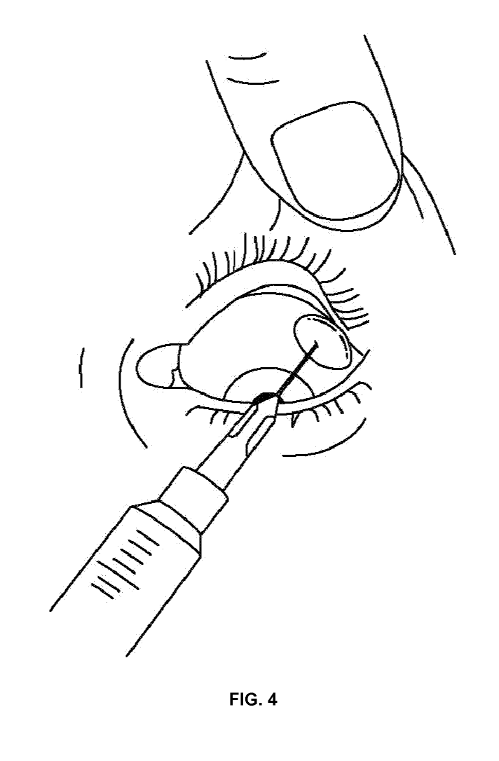

Referring to FIG. 3, the subconjunctival space around the eye can be divided into four quadrants: the superior, nasal, inferior, and temporal. These quadrants may be further subdivided into sub-quadrants, such as the superotemporal, superonasal, inferior nasal, and inferior temporal, and so on. Hence, the lipocalin muteins of the invention may be administered, for example, to the superotemporal quadrant of the bulbar subconjunctival space, or to any one or more of the inferior, superior, nasal, or temporal quadrants of the bulbar, palpepral, or forniceal subconjunctival spaces, or to the superotemporal, superonasal, inferior temporal, or inferior nasal bulbar, palpepral, or forniceal subconjunctival spaces (FIGS. 4-5). Therefore, unless further delimited, administration into the “subconjunctival space” of the eye refers to administration into any of the bulbar, palpepral, and/or forniceal subconjunctival spaces in the eye in any one or more of the four quadrants of the eye (superior, nasal, temporal, and inferior), or any one or more of the possible sub-quadrants of the eye, including the supertemporal, superonasal, inferior temporal, or inferior nasal regions of the bulbar, palpepral, or forniceal subconjunctival spaces.

“Neovascular diseases of the anterior segment of the eye” means those diseases of the anterior segment that are amenable to treatment by medication that inhibits angiogenesis. Such diseases include, for example, pterygia, corneal neovascularization, rubeosis iridis, toxic epidermal necrolysis, cicatrical permphigoid, pingueculitis, and dry eye.

A pterygium is a very common conjunctival degenerative condition. In the clinic, a pterygium is a pink, wedge-shaped growth extending from the inside corner of the eye towards the cornea, the clear dome covering the iris and pupil. Pterygia are benign, but can extend over the cornea, leading to obstruction of vision or astigmatism (corneal distortion). They can also cause chronic irritation or discomfort, a bloodshot appearance to the eye, and difficulty wearing contact lenses. They are accompanied by neovascularization, possibly occurring due to frequent exposure to ultra violet light, wind, and irritants. Currently there is no effective medication to treat pterygium, while pterygium surgery is the recommended treatment, with a high success rate, but also a likelihood of recurrence.

Invasion of new blood vessels into the normally avascular cornea occurs after infection and injury. Corneal neovascularization may be induced by a number of angiogenic growth factors. Inflammatory cells, such as macrophages and monocytes, also contain various angiogenic growth factors and corneal inflammation is a common stimulus for neovascularization. Corneal neovascularization is a challenging condition, and because corneal clarity and avascularity are critical for maintaining vision, developing treatments for corneal neovascularization is crucial. Corneal neovascularization occurs as a result of disequilibrium between angiogenic and antiangiogenic stimuli. Some causes of corneal neovascularization include, but are not limited to, trauma, aniridia, alkalai burn, interstitial keratitis, and ocular cicatricial pemphigoid.

Rubeosis iridis is a medical condition of the iris of the eye in which new abnormal blood vessels (i.e. neovascularization) are found on the surface of the iris. It is usually associated with disease processes in the retina, which involve the retina becoming starved of oxygen (ischaemic). The ischemic retina releases a variety of factors, the most important of which is vascular endothelial growth factor. These factors stimulate the formation of new blood vessels (angiogenesis). The new blood vessels can form in areas that do not have them. Specifically, new blood vessels can be observed on the iris. In addition to the blood vessels in the iris, they can grow into the angle of the eye. New blood vessels obstruct aqueous outflow leading to glaucoma. The neovascularization in the trabecular meshwork of the anterior chamber is observed in diabetes. Diffusible angiogenic factors, such as vascular endothelial growth factor are thought to originate from ischemic retinal tissues and promote neovascularization in the anterior chamber.

Therapeutic formulations of the lipocalins described here may be prepared by mixing the lipocalin having the desired degree of purity with optional physiologically acceptable carriers, excipients or stabilizers (Remington's Pharmaceutical Sciences 16th edition, Osol, A. Ed. (1980)), in the form of lyophilized formulations or aqueous solutions. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, 27dentate27es, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN®, PLURONICS®, or polyethylene glycol (PEG).

In relationship to any of the compositions described herein, it is preferable that an effective amount of buffer be included to maintain the pH from about 6 to about 8, preferably about 7. Buffers used are those known to those skilled in the art, and, while not intending to be limiting, some examples are acetate, borate, carbonate, citrate, and phosphate buffers. Preferably, the buffer comprises borate. An effective amount of buffer necessary for the purposes of this invention can be readily determined by a person skilled in the art without undue experimentation. In cases where the buffer comprises borate, it is preferable that the concentration of the borate buffer be about 0.6%.

In any of the compositions related described herein related to this invention, it is preferable for a tonicity agent to be used. Tonicity agents are used in ophthalmic compositions to adjust the concentration of dissolved material to the desired isotonic range. Tonicity agents are known to those skilled in the ophthalmic art, and, while not intending to be limiting, some examples include glycerin, mannitol, sorbitol, sodium chloride, and other electrolytes. Preferably, the tonicity agent is sodium chloride.

In any of the compositions related to the present invention which are described herein, it is preferable for a preservative to be used when the composition is intended for multiple use. There may also be reasons to use a preservative in single use compositions depending on the individual circumstances. The term preservative has the meaning commonly understood in the ophthalmic art. Preservatives are used to prevent bacterial contamination in multiple-use ophthalmic preparations, and, while not intending to be limiting, examples include benzalkonium chloride, stabilized oxychloro complexes (otherwise known as Purite®), phenylmercuric acetate, chlorobutanol, benzyl alcohol, parabens, and thimerosal. Preferably, the preservative is benzalkonium chloride (BAK).

Under certain circumstances, a surfactant might be used in any of the compositions related to this invention which are described herein. The term surfactant used herein has the meaning commonly understood in the art. Surfactants are used to help solubilize the therapeutically active agent or other insoluble components of the composition, and may serve other purposes as well. Anionic, cationic, amphoteric, zwitterionic, and nonionic surfactants may all be used in this invention. For the purposes of this invention, it is preferable that a nonionic surfactant, such as polysorbates, poloxamers, alcohol ethoxylates, ethylene glycol-propylene glycol block copolymers, fatty acid amides, alkylphenol ethoxylates, or phospholipids, is used in situations where it is desirable to use a surfactant.

Another type of compound that might be used in any composition of this invention described herein is a chelating agent. The term chelating agent refers to a compound that is capable of complexing a metal, as understood by those of ordinary skill in the chemical art. Chelating agents are used in ophthalmic compositions to enhance preservative effectiveness. While not intending to be limiting, some useful chelating agents for the purposes of this invention are 29dentate salts, like 29dentate disodium, 29dentate calcium disodium, 29dentate sodium, 29dentate trisodium, and 29dentate dipotassium.

The formulations can be sterilized by numerous means, including filtration through a bacteria-retaining filter, or by incorporating sterilizing agents in the form of sterile solid compositions which can be dissolved or dispersed in sterile water or other sterile medium just prior to use.

What is claimed is:

1. A method of treating a neovascular disease of the anterior segment of the human eye, the method comprising topically administering to an eye a mutein of human tear lipocalin, wherein the mutein comprises at least 12-16 amino acid mutations with respect to the wild type amino acid sequence of mature human tear lipocalin as set forth in SEQ ID NO: 1, wherein the mutations are selected from any of amino acids 25, 26, 27, 28, 29, 30, 31, 32, 33, 56, 57, 58, 83, 105, 106, 108 and 109, and wherein the mutein possesses at least 80% sequence identity with SEQ ID NO: 1 and binds VEGF with detectable affinity.

2. The method of claim 1, wherein the mutein comprises at least any 16 amino acid mutations at any of the sequence positions 25, 26, 27, 28, 29, 30, 31, 32, 33, 56, 57, 58, 83, 105, 106, 108 and 109 of the linear polypeptide sequence of the mature wild-type form of human tear lipocalin set forth in SEQ ID NO: 1.

3. The method of claim 1, further comprising 12-16 additional amino acid mutations selected from any of amino acids 8, 9, 10, 11, 12, 13, 43, 45, 47, 70, 72, 74, 75, 90, 92, 94, and 97.

4. A method of treating a neovascular disease of the anterior segment of the human eye, the method comprising topically administering to an eye a mutein of human tear lipocalin, wherein the mutein comprises at least 12-16 amino acid mutations with respect to the wild type amino acid sequence of mature human tear lipocalin, wherein the mutations are selected from any of the amino acids 25, 26, 27, 28, 29, 30, 31, 32, 33, 56, 57, 58, 83, 105, 106, 108 and 109 of the linear polypeptide sequence of the mature wild-type form of human tear lipocalin set forth in SEQ ID NO: 1, and wherein the mutein binds VEGF with detectable affinity, wherein the mutein possesses at least 70% sequence identity with SEQ ID NO: 1, wherein sequence identity means the percentage of pair-wise identical residues, following homology alignment of a sequence of a polypeptide with a sequence in question, with respect to the number of residues in the longer of these two sequences.

5. The method of claim 4, wherein the mutein comprises at least 16 amino acid mutations at any of the sequence positions 25, 26, 27, 28, 29, 30, 31, 32, 33, 56, 57, 58, 83, 105, 106, 108 and 109 of the linear polypeptide sequence of the mature wild-type form of human tear lipocalin set forth in SEQ ID NO: 1.

6. The method of claim 4, further comprising 12-16 additional amino acid mutations selected from any of the amino acids 8, 9, 10, 11, 12, 13, 43, 45, 47, 70, 72, 74, 75, 90, 92, 94, and 97 of the linear polypeptide sequence of the mature wild-type form of human tear lipocalin set forth in SEQ ID NO: 1.

7. The method of claim 1, wherein the disease is ptyregia.

8. The method of claim 1, wherein the disease is corneal neovascularization.

9. The method of claim 1, wherein the disease is rubeosis iridis.

10. The method of claim 1, wherein the disease is dry eye.
